(19) United States
(12) Patent Application Publication
(10) Pub. No.: US 2015/0196400 A1
(43) Pub. Date: Jul. 16, 2015

(54) SPINAL IMPLANT SYSTEM AND METHOD OF USE

(71) Applicant: Warsaw Orthopedic, Inc., Warsaw, IN (US)

(72) Inventor: Mark C. Dace, Collierville, TN (US)

(73) Assignee: Warsaw Orthopedic, Inc., Warsaw, IN (US)

(21) Appl. No.: 14/155,076

(22) Filed: Jan. 14, 2014

(51) Int. Cl. A61F 2/44

(52) U.S. Cl. CPC A61F 2/4455; A61F 2002/30168

(57) ABSTRACT

A spinal implant comprises an interbody portion including a first endplate engaging surface and a second endplate engaging surface. The portion defines at least one first cavity oriented to implant a fastener with endplate tissue. A plate portion extends from the interbody portion in a substantially perpendicular orientation and defines at least one second cavity oriented to implant a fastener with vertebral wall tissue. Systems and methods are disclosed.

### TECHNICAL FIELD

The present disclosure generally relates to medical devices for the treatment of musculoskeletal disorders, and more particularly to a surgical system and method for treating a spine.

### BACKGROUND

Spinal disorders such as degenerative disc disease, disc herniation, osteoporosis, spondylolisthesis, stenosis, scoliosis and other curvature abnormalities, kyphosis, tumor, and fracture may result from factors including trauma, disease and degenerative conditions caused by injury and aging. Spinal disorders typically result in symptoms including pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes discectomy, corpectomy, laminectomy, fusion, fixation and implantable prosthetics. As part of these surgical treatments, implants, such as, for example, spinal constructs including plates, rods, fasteners or interbody devices are often employed for stabilization of a treated section of a spine. This disclosure describes an improvement over these prior art technologies.

### SUMMARY

In one embodiment, a spinal implant is provided. The spinal implant comprises an interbody portion including a first endplate engaging surface and a second endplate engaging surface. The portion defines at least one first cavity oriented to implant a fastener with endplate tissue. A plate portion extends from the interbody portion in a substantially perpendicular orientation and defines at least one second cavity oriented to implant a fastener with vertebral wall tissue. Systems and methods are disclosed.

### BRIEF DESCRIPTION OF THE DRAWINGS

The present disclosure will become more readily apparent from the specific description accompanied by the following drawings, in which:

FIG. 1 is a perspective view of components of one embodiment of a system in accordance with the principles of the present disclosure;

FIG. 2 is a side view of components of one embodiment of a system in accordance with the principles of the present disclosure disposed with vertebrae;

FIG. 3 is a plan view of the components and vertebrae shown in FIG. 2:

FIG. 4 is a plan view of components of one embodiment of a system in accordance with the principles of the present disclosure disposed with vertebrae;

FIG. 5 is an enlarged view of the components shown in detail AA in FIGS. 4; and

FIG. 6 is a side view of components of one embodiment of a system in accordance with the principles of the present disclosure disposed with vertebrae.

### DETAILED DESCRIPTION

The exemplary embodiments of the surgical system and related methods of use disclosed are discussed in terms of medical devices for the treatment of musculoskeletal disorders and more particularly, in terms of a surgical system and a method for treating a spine. In one embodiment, the systems and methods of the present disclosure are employed with a spinal joint fusion, for example, with a cervical, thoracic, lumbar and/or sacral region of a spine. In one embodiment, a spinal implant system is provided that comprises a spinal construct having an interbody portion, a plate portion and/or bone fasteners. In one embodiment, the spinal implant system includes a spinal implant that is delivered and/or introduced with an anterior surgical approach. In one embodiment, the spinal implant is delivered and/or introduced with an oblique lateral surgical approach.

In one embodiment, the presently disclosed system is employed with an anterior lumbar interbody fusion (ALIF) procedure adjacent to vertebrae that has been previously instrumented with an anterior cervical plate. In one embodiment, the presently disclosed system is employed with a method including an oblique lateral interbody fusion (OLIF) procedure.

In one embodiment, the presently disclosed system is employed with a method including a surgical procedure for treating intervertebral disc spaces that are adjacent to existing fusions to alleviate a patients painful symptoms, In some embodiments, the present system includes a spinal implant comprising an interbody portion and a plate portion, which avoids overlap with an existing plate construct. In some embodiments, the spinal implant comprises a stand-alone interbody device that is utilized with an existing plate construct.

In one embodiment, the spinal implant includes an anterior bone screw fixation of the plate portion. In one embodiment, the spinal implant includes an inferior side of the interbody portion and the plate portion allows for a single screw into a vertebral endplate to minimize interference with screws from a previously implanted plate, In one embodiment, the spinal implant includes a superior side of the interbody portion and the plate portion allows for two bone screws attached to a sidewall of a superior vertebral body to provide stability. In one embodiment, the spinal implant has a standalone configuration such that the interbody portion and the plate portion comprise an L-shaped configuration. In one embodiment, the spinal implant comprises a half-flange plate configuration.

In one embodiment, the spinal implant comprises the interbody portion having at least one inter-discal bone screw hole and the plate portion having at least one anterior bone screw hole. In one embodiment, the spinal implant avoids anterior vertebral surface coverage on the side of the adjacent existing plate. In one embodiment, the spinal implant provides for anterior vertebral surface coverage fixation along an adjacent vertebral surface opposite an existing plate. In one embodiment, the spinal implant provides for anterior vertebral surface screw fixation with an adjacent vertebral surface opposite an existing plate.

In one embodiment, the spinal implant allows for insertion of bone screws along a trajectory at various angles when anatomical positioning prevents insertion of the screw at a large angle trajectory. In one embodiment, the spinal implant includes openings oriented to implant bone screws at converging and/or diverging trajectories.

The present disclosure may be understood more readily by reference to the following detailed description of the disclosure taken in connection with the accompanying drawing figures, which form a part of this disclosure. It is to be understood that this disclosure is not limited to the specific devices, methods, conditions or parameters described and/or shown herein, and that the terminology used herein is for the purpose of describing particular embodiments by way of example only and is not intended to be limiting of the claimed disclosure. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, vessels, ligaments, tendons, cartilage and/or bone unless specifically referred to otherwise.

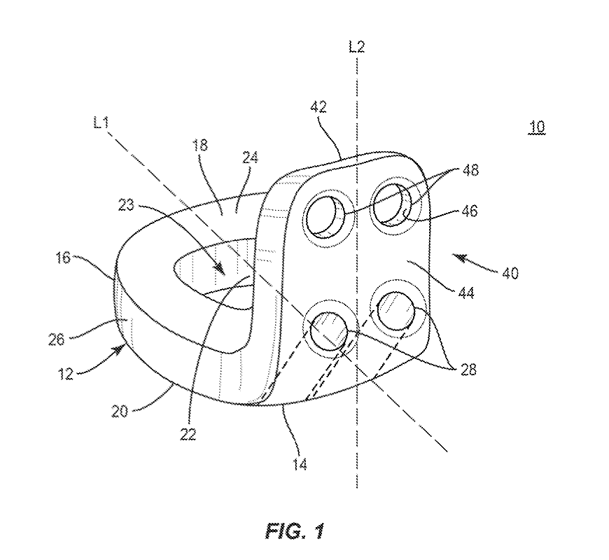

The following discussion includes a description of a system including spinal implants, related components and methods of employing the system in accordance with the principles of the present disclosure. Alternate embodiments are also disclosed. Reference is made in detail to the exemplary embodiments of the present disclosure, which are illustrated in the accompanying figures. Turning to FIGS. 1-3, there are illustrated components of a surgical system, such as, for example, a spinal implant system including a spinal implant 10.

The components of the spinal implant system can be fabricated from biologically acceptable materials suitable for medical applications, including metals, synthetic polymers, ceramics and bone material and/or their composites. For example, the components of the spinal implant system, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO⁴ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of the spinal implant system may have material composites, including the above materials, to achieve various desired characteristics such as strength, rigidity, elasticity, compliance, biomechanical performance, durability and radiolucency or imaging preference. The components of the spinal implant system, individually or collectively, may also be fabricated from a heterogeneous material such as a combination of two or more of the above-described materials. The components of the spinal implant system may be monolithically formed, integrally connected or include fastening elements and/or instruments, as described herein. In one embodiment, a spinal implant, as described herein, may be formed substantially of biocompatible polymer, such as PEEK, and selectively coated with a biocompatible metal, such as titanium, or a bone-growth promoting material, such as HA. In some embodiments, titanium may be plasma sprayed onto surfaces of the spinal implant to modify a radiographic signature of the spinal implant and/or improve bony ongrowth to the spinal implant by application of a porous or semi-porous coating of titanium.

The spinal implant system may be employed, for example, with minimally invasive procedures, including percutaneous techniques, mini-open surgical techniques and/or open surgical techniques to deliver and introduce instrumentation and/or implants, such as, for example, spinal implant 10, at a surgical site within a subject body B of a patient, which includes, for example, a spine having vertebrae V. In some embodiments, the spinal implant system can include spinal constructs including one or more bone fasteners, spinal rods, connectors and/or plates. In some embodiments, various components of the spinal implant system may be utilized in open or traditional spinal surgical techniques. In some embodiments, a patient is positioned on their side for the surgical procedure and the surgeon may stand on an anterior side of the patient to be capable of standing directly above an oblique-anterior and/or an oblique lateral surgical pathway.

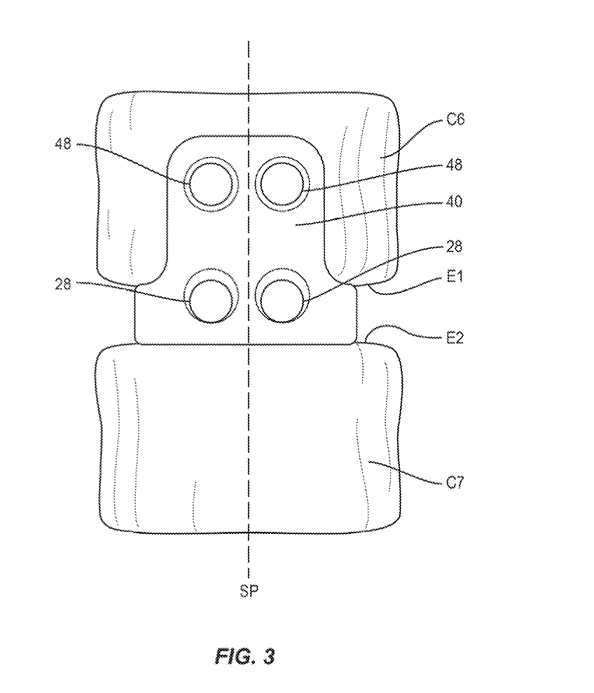

Spinal implant 10 includes an interbody portion, such as, for example, an interbody member 12. Interbody member 12 has an implant body that extends between an anterior surface 14 and a posterior surface 16. The implant body of member 12 defines a longitudinal axis L1. Anterior surface 14 defines an anterior face A and posterior surface 16 defines a posterior face P. In some embodiments, upon disposal of interbody member 12 with vertebrae, anterior face A is oriented to face an anterior side of body B and be disposed adjacent an anterior portion of vertebrae, such as, for example, an anterior portion Al of an intervertebral space of vertebrae V. In some embodiments, at least a portion of anterior face A defines an axis and/or plane substantially aligned with anterior portion Al upon disposal of interbody member 12 with vertebrae V, as shown in FIG. 3.

In some embodiments, upon disposal of interbody member 12 with vertebrae, posterior face P is oriented to face a posterior side of body B and be disposed adjacent a posterior portion of vertebrae, such as, for example, a posterior portion P1 of an intervertebral space of vertebrae V. In some embodiments, at least a portion of posterior face P defines an axis and/or plane substantially aligned with posterior portion. P1 upon disposal of interbody member 12 with vertebrae V.

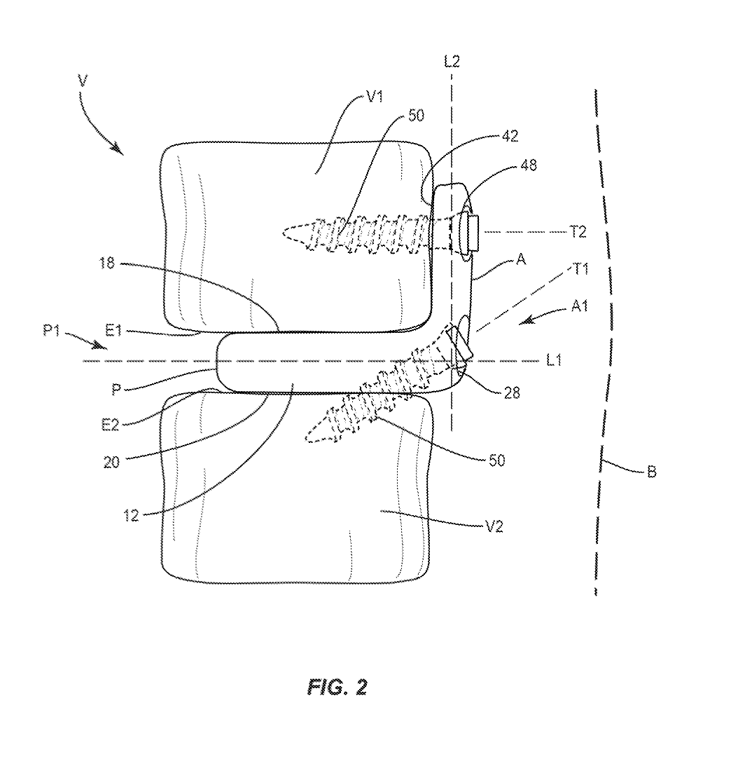

Interbody member 12 includes a vertebral engaging surface 18 and a vertebral engaging surface 20. Surface 18 is substantially planar and configured to engage endplate tissue of a vertebral body, such as, for example, an endplate E1, as shown in FIG. 2. Surface 20 is configured to engage endplate tissue of a vertebral body, such as, for example, an endplate E2. In some embodiments, surface 18 and/or surface 20 may be rough, textured, porous, semi-porous, dimpled, knurled, toothed, grooved and/or polished to facilitate engagement with tissue. In some embodiments, the vertebral tissue may include intervertebral tissue, endplate surfaces and/or cortical bone.

In one embodiment, interbody member 12 includes an inner surface 22 that defines an opening 23 configured to receive an agent, which may include bone graft (not shown) and/or other materials, as described herein, for employment in a fixation or fusion treatment. In some embodiments, the cross-sectional geometry of interbody member 12 may have various configurations, such as, for example, cylindrical, round, oval, oblong, triangular, polygonal having planar or arcuate side portions, irregular, uniform non-uniform, consistent, variable, horseshoe shape, U-shape or kidney bean shape.

Interbody member 12 includes an outer surface 24 that is continuous between anterior surface 14 and posterior surface 16. Interbody member 12 includes a side surface 26 that is continuous with surface 24. In one embodiment, surface 24 and/or surface 26 are smooth or even. In some embodiments, surface 24 and/or surface 26 may be textured, rough, porous, semi-porous, dimpled and/or polished.

Surface 22 defines at least one cavity, such as, for example, spaced through openings 28, as shown in FIG. 1 in phantom and in FIG. 2, oriented to implant a fastener with endplate tissue E2 along a trajectory T1. In one embodiment, trajectory T1 implants a fastener at an angle in a range of approximately 20 to 45 degrees relative to an axis L2 of a flange 40 and/or the surfaces of flange 40, as described herein. In one embodiment, trajectory T1 is disposed in a sagittal plane SP of vertebrae V, as shown in FIG. 3. In some embodiments, all or only a portion of surface 22 that defines openings 28 defines an internally threaded portion configured for threaded fixation with a fastener.

In some embodiments, upon disposal of interbody member 12 with vertebrae V, openings 28 are oriented with interbody member 12 in substantial alignment with an oblique surgical pathway formed with body B. In some embodiments, substantial alignment of all or only a portion of openings 28 with all or only a portion of the oblique surgical pathway includes co-axial, spaced apart, offset and/or angularly offset. In some embodiments, upon disposal of interbody member 12 with vertebrae V, openings 28 are oriented with interbody member 12 in substantial alignment with an anterior surgical pathway formed with body B. In some embodiments, substantial alignment of all or only a portion of openings 28 with all or only a portion of the anterior surgical pathway includes co-axial, spaced apart, offset and/or angularly offset.

Spinal implant 10 includes a plate portion, such as, for example, a flange 40 extending from interbody member 12 at an angular orientation. Flange 40 extends from member 12 to define an axis L2. As shown in FIG. 1, flange 40 extends from interbody member 12 such that axis L2 is disposed in a substantially perpendicular orientation relative to axis L1.

In some embodiments, interbody member 12 and flange 40 comprise a substantially L-shaped configuration. In some embodiments, the substantially perpendicular orientation of axes L1, L2 includes axis L2 being disposed at an angle relative to axis L1 in a range of 75 to 105 degrees to accommodate variations in patient anatomy. In some embodiments, the substantially perpendicular orientation of axes L1, L2 includes axis 12 being disposed at an angle relative to axis L1 in a range of −15 to 15 degrees relative to a perpendicular orientation of 90 degrees. In some embodiments, the orientation of axes L1, L2 includes axis L2 being disposed at a perpendicular orientation of 90 degrees relative to axis L1. In some embodiments, axes L1, L2 are disposed in a co-axial, spaced apart, offset and/or angularly offset configuration. In some embodiments, interbody member 12 and flange 40 are disposed in a co-axial, spaced apart, offset and/or angularly offset configuration. In one embodiment, interbody member 12 and flange 40 are monolithically formed. In some embodiments, interbody member 12 and flange 40 may be integrally connected, separate and attachable or include fastening elements for attachment. In some embodiments, flange 40 may be attached with interbody member 12 prior to implantation or in situ.

Flange 40 includes a posterior facing surface, such as, for example, a vertebra engaging surface 42 and an anterior facing surface, such as, for example, an outer surface 44. In some embodiments, surface 42 and/or surface 44 may have various configurations, such as, for example, planar, irregular, uniform, non-uniform, consistent, and variable In some embodiments, surface 42 and/or surface 44 may be textured, rough, porn s semi-porous, dimpled and/or polished to facilitate or prevent movement and/or rotation.

Surface 42 is continuous with surface 18 such that surface 42 forms an arcuate configuration with surface 18. In some embodiments, surface 42 and surface 18 are disposed in a spaced apart, offset and/or angularly offset configuration. In one embodiment, surface 44 is continuous with surface 24 such that surface 44 is in a substantially perpendicular orientation with surface 24. In some embodiments, surface 44 and surface 24 are disposed in a spaced apart, offset and/or angularly offset configuration.

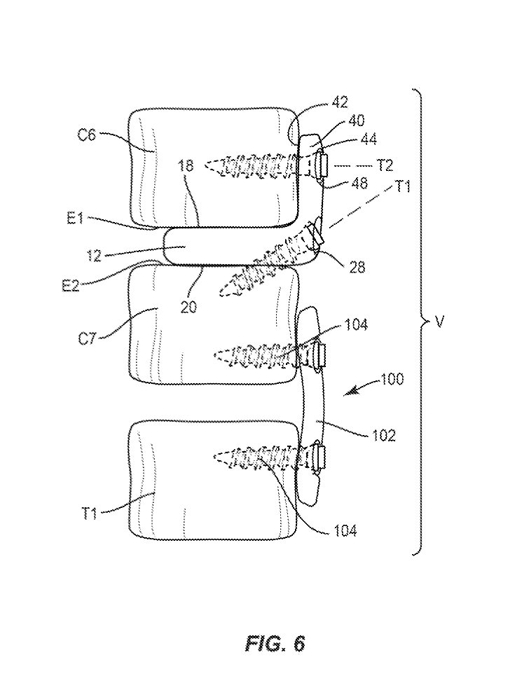

Flange 40 includes an inner surface 46. Surface 46 defines at least one cavity, such as, for example, spaced through openings 48, as shown in FIG. 1 in phantom and in FIG. 2, oriented to implant a fastener with a wall of a vertebral body, such as, for example, vertebrae V1 along a second trajectory T2. In some embodiments, trajectory T2 implants a fastener at an angle in a range of approximately −5 to 35 degrees relative to axis L2 of flange 40 and/or the surfaces of flange 40, as described herein. In one embodiment, trajectory T2 is disposed in sagittal plane SP of vertebrae V and transverse to trajectory T1. In some embodiments, all or only a portion of surface 46 that defines openings 48 defines an internally threaded portion configured for threaded fixation with a fastener. In one embodiment, trajectory T2 implants the fastener in an orientation to resist and/or prevent interference with a preexisting spinal construct implanted with adjacent vertebrae, for example, spinal construct 100, as shown in FIG. 6. Trajectory T2 facilitates engagement and fixation of a fastener at vertebral level V1 to avoid and/or modify trajectory T1 into the shared vertebral level V2 with spinal construct 100.

In some embodiments, upon disposal of flange 40 with vertebrae V, openings 48 are oriented with flange 40 in substantial alignment with an oblique surgical pathway formed with body B. In some embodiments, substantial alignment of all or only a portion of openings 48 with all or only a portion of the oblique surgical pathway includes co-axial, spaced apart, offset and/or angularly offset. In some embodiments, upon disposal of flange 40 with vertebrae V, openings 48 are oriented with flange 40 in substantial alignment with an anterior surgical pathway formed with body B. In some embodiments, substantial alignment of all or only a portion of openings 48 with all or only a portion of the anterior surgical pathway includes co-axial, spaced apart, offset and/or angularly offset.

The spinal implant system includes one or more fasteners, such as, for example, bone screws 50, as shown in FIG. 2, for attaching spinal implant 10 including interbody member 12 and/or flange 40 with tissue, as described herein. In some embodiments, bone screws 50 may be engaged with tissue in various orientations, such as, for example, series, parallel, offset, staggered and/or alternate vertebral levels. In some embodiments, one or more of bone screws 50 may comprise multi-axial screws, sagittal angulation screws, pedicle screws, mono-axial screws, uniplanar screws, facet screws, fixed screws, tissue penetrating screws, conventional screws, expanding screws, wedges, anchors. buttons, clips, snaps, friction fittings, compressive fittings, expanding rivets, staples, nails, adhesives, fixation plates and/or post.

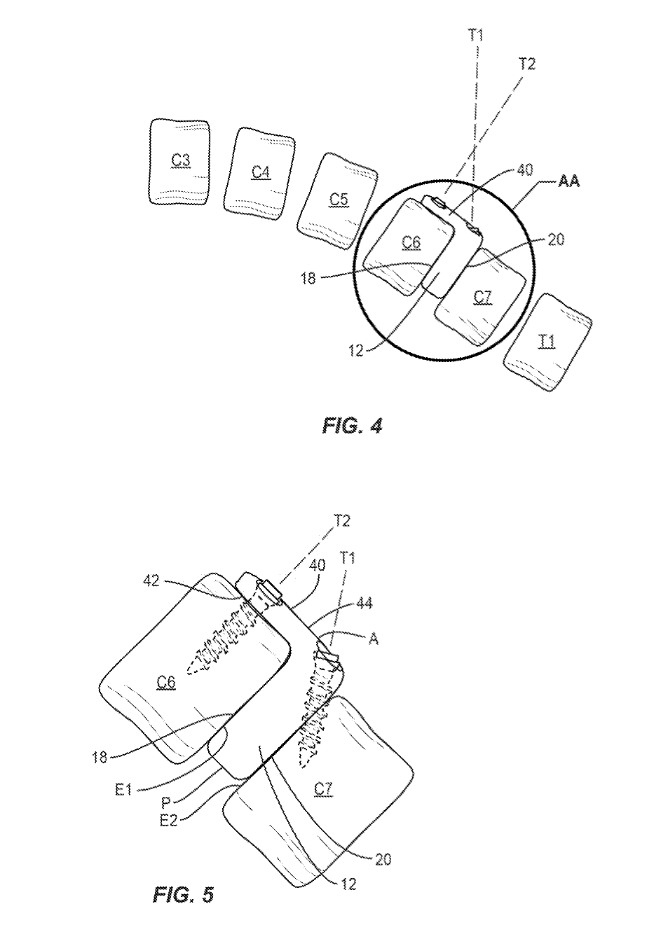

In one embodiment, as shown in FIGS. 4 and 5, the spinal implant system, similar to the systems and methods described herein, which includes spinal implant 10, is employed with a particular surgical procedure, such as, for example, a cervical joint fusion for treating cervical vertebral levels and/or adjacent thoracic vertebral levels,

To treat the affected section of vertebrae V, disposed at a selected surgical site, such as, for example, vertebrae C3-T1, an incision is made with a surgical instrument, such as, for example, a scalpel, for substantial alignment and communication with a selected surgical pathway. In some embodiments, the spinal implant system can be employed with an ALIF procedure or an OLIF procedure, as described herein.

In some embodiments, a discectomy is performed adjacent the intervertebral space. In some embodiments, sequential trial implants are delivered along the surgical pathway and used to distract the intervertebral space and apply appropriate tension in the intervertebral space allowing for indirect decompression. In some embodiments, the size of spinal implant 10 including interbody member 12 is selected after trialing. Spinal implant 10 is visualized by fluoroscopy and oriented before malleting interbody member 12 into the intervertebral space.

An inserter (not shown) is connected with interbody member 12 and/or flange 40 to guide and facilitate delivery of spinal implant 10 into body B along the selected surgical pathway. The inserter delivers spinal implant 10 through the incision along the surgical pathway adjacent to the selected surgical site for implantation into an intervertebral space between vertebrae C6, C7. Interbody member 12 is disposed with the intervertebral space between vertebrae C6, C7. Surface 18 is disposed in a cephalad orientation of body B and engages endplate E1 of vertebrae C6. Surface 20 is disposed in a caudal orientation of body B and engages endplate E2 of vertebrae C7.

Spinal implant 10 is disposed such that anterior face A is oriented to face the anterior side of body B and be disposed adjacent anterior portion A1 (for example, as shown in FIG. 2), Posterior face P is oriented to face the posterior side of body B and be disposed adjacent posterior portion P1 (for example, as shown in FIG. 2). Openings 28 are oriented to implant bone screws 50 with endplate E2 along trajectory T1 such that interbody member 12 is fixed with the intervertebral space.

Flange 40 is positioned such that surface 42 engages the wall surface of vertebra C6 and surface 44 faces anterior side A1. Openings 48 are oriented to implant bone screws 50 with the wall of vertebra C6 along trajectory T2. Bone screws 50 are inserted through openings 28 along trajectory T1 to engage and fix interbody member 12 with endplate E2 and the vertebrae. Bone screws 50 are inserted through openings 48 along trajectory T2 to engage and fix flange 40 with vertebra C6.

In one embodiment, as shown in FIG. 6, a previously implanted spinal construct 100 is disposed with vertebrae C7 and T1. Spinal construct 100 may include, such as, for example, a plate 102 and fasteners 104. Plate 102 comprises instrumentation from a prior procedure and extends along a portion of vertebra C7 thereby reducing the usable surface area of a wall of vertebra C7 for subsequent procedures to attach a spinal implant to vertebra C7. Fastener 104 extends into vertebrae C7 thereby reducing a distance into which bone screw 50 of interbody member 12 or flange 40 can extend into vertebra C7.

Spinal construct 100 is disposed with vertebrae C6 and C7. Trajectory T1 is oriented such that bone screw 50 is inserted along trajectory T1 to securely fasten interbody member 12 with vertebrae C7 and avoid interference with spinal construct 100. Bone screw 50 is inserted through opening 28 along trajectory T1 such that bone screw 50 avoids interference with fasteners 104.

Upon completion of a procedure, as described herein, the surgical instruments, assemblies and non-implanted components of the spinal implant system are removed and the incision(s) are closed. One or more of the components of the spinal implant system can be made of radiolucent materials such as polymers. Radiopaque markers may be included for identification under x-ray, fluoroscopy, CT or other imaging techniques. In some embodiments, the use of surgical navigation, microsurgical and image guided technologies may be employed to access, view and repair spinal deterioration or damage, with the aid of the spinal implant system. In some embodiments, the spinal implant system may include one or a plurality of plates, connectors and/or bone fasteners for use with a single vertebral level or a plurality of vertebral levels.

In one embodiment, the spinal implant system includes an agent, for example, as described above, which may be disposed, packed, coated or layered within, on or about the components and/or surfaces of the spinal implant system. In some embodiments, the agent may include bone growth promoting material, such as, for example, bone graft to enhance fixation of the components and/or surfaces of the spinal implant system with vertebrae. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A spinal implant comprising:
an interbody portion including a first endplate engaging surface and a second endplate engaging surface, the portion defining at least one first cavity oriented to implant a fastener with endplate tissue; and
a plate portion extending from the interbody portion in a substantially perpendicular orientation and defining at least one second cavity oriented to implant a fastener with vertebral wall tissue.

2. A spinal implant as recited in claim 1 wherein the interbody portion extends between an anterior surface and a posterior surface such that an outer surface of the interbody portion is continuous therebetween.

3. A spinal implant as recited in claim 1 wherein the interbody portion includes side surfaces such that an outer surface of the interbody portion is continuous therebetween.

4. A spinal implant as recited in claim 1, wherein the at least one first cavity includes a pair of spaced through openings.

5. A spinal implant as recited in claim 1, wherein the plate o on includes a flange.

6. A spinal implant as recited in claim 1, wherein the at least one second cavity includes a pair of spaced through openings.

7. A spinal implant as recited in claim 1, wherein the interbody portion and the plate portion comprise an L-shaped configuration.

8. A spinal implant as recited in claim 1, wherein the interbody portion and the plate portion are monolithically formed.

9. A spinal implant as recited in claim 1, wherein the at least one first cavity implants a fastener along a first trajectory disposed in a sagittal plane of vertebrae and the at least one second cavity implants a fastener along a second trajectory disposed in the sagittal plane and disposed transverse to the first trajectory.

10. A spinal implant as recited in claim 9, wherein at least one of the first trajectory and the second trajectory is substantially aligned with a substantially anterior surgical pathway.

11. A spinal implant as recited in claim 9, wherein at least one of the first trajectory and the second trajectory is substantially aligned with a substantially oblique surgical pathway.

12. A spinal implant as recited in claim 1, wherein the first endplate engaging surface is continuous with a vertebral engaging surface of the plate such that the first endplate engaging surface and the vertebral engaging surface comprise an arcuate configuration.

13. A spinal implant as recited in claim 1, wherein the second endplate engaging surface is continuous with an outer surface of the plate such that the second endplate engaging surface and the outer surface comprise the substantially perpendicular orientation.

14. A spinal implant as recited in claim 1, wherein the at least one second cavity implants the fastener to resist and/or prevent interference with a preexisting spinal construct implanted with an adjacent vertebrae.

15. A spinal implant as recited in claim 1, wherein the at least one first cavity implants a fastener at an angle in a range of approximately 20 to 45 degrees relative to an axis of the plate portion.

16. A spinal implant as recited in claim 1 wherein the at least one second cavity implants a fastener at an angle in a range of approximately −5 to 35 degrees relative to an axis of the plate portion.

17. A spinal implant comprising:
an interbody member extending between an anterior surface and a posterior surface and including a first surface configured to engage endplate tissue of a first vertebrae and a second surface configured to engage endplate tissue of a second vertebrae, the member including an inner surface that defines at least one through opening oriented to implant a fastener with the endplate tissue of the second vertebrae along a first trajectory disposed in a sagittal plane of the vertebrae, and
a flange extending from the member in a substantially perpendicular orientation and defining an inner surface that defines at least one through opening oriented to implant a fastener with a wall of the first vertebrae along a second trajectory disposed in the sagittal plane and transverse to the first trajectory.

18. A spinal implant as recited in claim 17, wherein at least one of the first trajectory and the second trajectory is substantially aligned with a substantially anterior surgical pathway.

19. A spinal implant as recited in claim 17, wherein at least one of the first trajectory and the second trajectory is substantially aligned with a substantially oblique surgical pathway.

20. A spinal implant system comprising:
at least one first bone screw;
at least one second bone screw; and
an interbody implant comprising an interbody portion including a t endplate engaging surface and a second endplate engaging surface, the portion defining at least one first cavity oriented to implant the at least one first bone screw with endplate tissue along a first trajectory, and a plate portion extending from the interbody portion in a substantially perpendicular orientation and defining at least one second cavity oriented to implant the at least one second bone screw with vertebral wall tissue along a second trajectory disposed transverse to the first trajectory.
